(19) United States
(12) Patent Application Publication
(10) Pub. No.: US 2002/0183242 A1
(43) Pub. Date: Dec. 5, 2002

(54) Peptide antiangiogenic drugs

(76) Inventors: Jack Henkin, Highland Park, IL (US); Fortuna Haviv, Deerfield, IL (US); Michael F. Bradley, Wadsworth, IL (US); Douglas M. Kalvin, Buffalo Grove, IL (US); Andrew J. Schneider, Gurnee, IL (US)

Correspondence Address: Steven F. Weinstock, Abbott Laboratories, Dept. 377 Bldg. AP6D-2, 100 Abbott Park Road, Abbott Park, IL, 60064-6050, US

(21) Appl. No.: 09/832,733

(22) Filed: Apr. 11, 2001

(51) Int. Cl. C07K 17/00; C07K 16/00; C07K 14/00; C07K 7/00; C07K 5/00; C07K 4/00; C07K 2/00; A61K 38/00; A01N 37/18

(52) U.S. Cl. 514/2; 530/300

(57) ABSTRACT

The present invention discloses peptides which are useful for inhibiting angiogenesis. Also disclosed are angiogenesis-inhibiting compositions and methods of inhibiting angiogenesis in a mammal.

### TECHNICAL FIELD

The invention relates to novel compounds having activity useful for treating conditions which arise or are exacerbated by angiogenesis, pharmaceutical compositions comprising the compounds, methods of treating using the compounds, and methods of inhibiting angiogensis.

### BACKGROUND OF THE INVENTION

Angiogenesis, the process by which new blood vessels are formed, is essential for normal body activities including reproduction, development, and wound repair. Although the process is not completely understood, it is believed to involve a complex interplay of molecules which regulate the growth of endothelial cells (the primary cells of capillary blood vessels). Under normal conditions, these molecules appear to maintain the microvasculature in a quiescent state (i.e., one of no capillary growth) for prolonged periods which may last for weeks or, in some cases, decades. When necessary (such as during wound repair), these same cells can undergo short bursts of growth and rapid proliferation (J. Biol. Chem. 1992, 267, 10931-10934, and Science 1987, 235, 442-447.

While it is normally a regulated process, many diseases (characterized as angiogenic diseases) are driven by persistent, unregulated angiogenesis. Ocular neovascularization has been implicated as the most common cause of blindness and is responsible for approximately twenty different eye diseases. In certain existing conditions, such as arthritis, newly formed capillary blood vessels invade the joints and destroy cartilage. The growth and metastasis of solid tumors are also dependent on angiogenesis (Cancer Res. 1986, 46, 467-473, and J. Natl. Cancer Inst. 1989, 82, 4-6). It has been shown that solid tumors cannot grow beyond 1 to 2 cubic millimeters without inducing the formation of new blood vessels. Once these new blood vessels become embedded in the tumor, they provide a means for tumor cells to enter the circulation and metastasize to distant sites such as the liver, the lungs, or the bones (N. Engl. J. Med. 1991, 324, 1-8).

Several angiogenesis inhibitors are currently under development for use in treating angiogenic diseases, but there are disadvantages associated with these compounds. Fumagillin, a compound secreted by the fungus Aspergillus fumigatis fresenius, has demonstrated angioinhibitory effects, but has not been developed clinically due to the dramatic weight loss suffered by laboratory animals after prolonged exposure. TNP-470, a synthetic analog of fumagillin, also inhibits endothelial growth, but has been shown to induce asthenial and neurocortical toxicity in humans, limiting allowable dosages (J. Clin. Oncology 1999, 17, 2541). Numerous peptide angiogenesis inhibitors have also been described (see, for example, WO99/61476, U.S. Pat. Nos. 5,932,545; 5,840,692; 5,426,100; and 5,190,918). However, there is still a need for compounds useful in treating angiogenic diseases which have improved profiles of activity. More specifically, there is a need for angiogenesis inhibitors which are safe for therapeutic use and which exhibit selective toxicity with respect to the pathological condition such as by selectively inhibiting the proliferation of endothelial cells while exhibiting no or a low degree of toxicity to normal (i.e. non-cancerous) cells. Such compounds should also be easily and cost-effectively made.

### SUMMARY OF THE INVENTION

In its principle embodiment, the invention provides a compound of formula (I)

N-Ac-Sar-Gly-AA³-AA⁴-AA⁵-AA⁶-AA⁷-Arg-Pro-AA¹⁰ (I),

or a pharmacutically acceptable salt, ester, prodrug, or solvate thereof, wherein

AA³ is selected from the group consisting of

(1) glutaminyl,

(2) phenylalanyl,

(3) valyl, and

(4) asparaginyl;

AA⁴ is selected from the group consisting of

(1) D-isoleucyl,

(2) isoleucyl,

(3) D-leucyl, and

(4) D-alloisoleucyl;

AA⁵is selected from the group consisting of

(1) seryl,

(2) methionyl,

(3) allothreonyl,

(4) threonyl, and

(5) tyrosyl;

AA⁶ is selected from the group consisting of

(1) norvalyl,

(2) seryl,

(3) tryptophyl,

(4) glutaminyl, and

(5) prolyl;

AA⁷ is selected from the group consisting of

(1) isoleucyl,

(2) D-isoleucyl,

(3) lysyl(acetyl), and

(4) prolyl; and

AA¹⁰ is selected from the group consisting of

(1) D-alanylamide,

(2) ethylamide, and

(3) isopropylamide;

with the proviso that one of AA⁴ and AA⁷ is a D-amino acid.

In another embodiment, the invention provides a pharmaceutical composition comprising a compound of formula (I), or a pharmacutically acceptable salt, ester, prodrug, or solvate thereof, and a pharmaceutically acceptable carrier.

In another embodiment, the invention provides a method of treating a patient in need of anti-angiogenesis therapy comprising administering to the patient in need a therapeutically effective amount of a compound of formula (I), or a pharmacutically acceptable salt, ester, prodrug, or solvate thereof.

In another embodiment, the invention provides a composition for the treatment of a disease selected from cancer, arthritis, psoriasis, angiogenesis of the eye associated with infection or surgical intervention, macular degeneration and diabetic retinopathy comprising a compound of formula (I), or a pharmaceutically acceptable salt thereof, in combination with a pharmaceutically acceptable carrier.

In another embodiment, the invention provides a method of isolating a receptor from an endothelial cell comprising binding a compound of formula (I), or a pharmacutically acceptable salt, ester, prodrug, or solvate thereof, to the receptor to form a peptide receptor complex; isolating the peptide receptor complex; and purifying the receptor.

### DETAILED DESCRIPTION OF THE INVENTION

Definition of Terms

As used herein, the singular forms “a”, “an”, and “the” include plural reference unless the context clearly dictates otherwise.

As used in the present specification the following terms have the meanings indicated:

The term “nicotinyl,” as used herein refers to the acyl group derived from nicotinic acid, i.e., pyridine-3-carboxylic acid.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

The term “pharmaceutically acceptable salt,” as used herein, refers to salts or zwitterionic forms of the compounds of the instant invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The term “pharmaceutically acceptable solvate,” as used herein, represents an aggregate that comprises one or more molecules of the solute, such as a formula (I) compound, with one or more molecules of solvent.

The term “receptor,” as used herein, refers to a chemical group or molecule on the cell surface or in the cell interior that has an affinity for a specific chemical group, molecule, or virus. Isolation of receptors relevant to the antiangiogenic activity of the peptide of the invention can provide useful diagnostic tools.

Unless indicated otherwise by a “D” prefix, e.g. D-Ala or D-Ile, the stereochemistry of the α-carbon of the amino acids and aminoacyl residues in peptides described in this specification and the appended claims is the natural or “L” configuration. The Cahn-Ingold-Prelog “R” and “S” designations are used to specify the stereochemistry of chiral centers in certain of the acyl substituents at the N-terminus of the peptides of this invention. The designation “R,S” is meant to indicate a racemic mixture of the two enantiomeric forms. This nomenclature follows that described in R. S. Cahn, et al., Angew. Chem. Int. Ed. Engl., 5, 385-415 (1966).

For the most part, the names on naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table 1.

TABLE 1
Amino Acid Abbreviations

| Abbreviation | Amino Acid |
|---|---|
| N-Ac-Sar | N-acetylsarcosyl |
| AlaNH₂ | alanylamide |
| alloIle | alloisoleucyl |
| alloThr | allothreonyl |
| alloThr (t-Bu) | allothreonyl (O-t-butyl) |
| Arg | arginyl |
| Arg (Pmc) | (N^{G}-2,2,5,7,8-pentamethylchroman-6- |
| | sulfonyl) arginyl |
| Asn | asparaginyl |
| Asn (Trt) | asparaginyl (trityl) |
| Gln | glutaminyl |
| Gln (Trt) | glutaminyl (trityl) |
| Gly | glycyl |
| Ile | isoleucyl |
| Leu | leucyl |
| Lys (Ac) | lysyl (N-epsilon-acetyl) |
| 6-Me-Nicotinyl | 6-methylnicotinyl |
| Met | methionyl |
| Nva | norvalyl |
| Phe | phenylalanyl |
| Pro | prolyl |
| Sar | sarcosyl |
| Ser | seryl |
| Ser (t-Bu) | seryl (O-t-butyl) |
| Thr | threonyl |
| Thr (t-Bu) | threonyl (O-t-butyl) |
| Trp | tryptyl |
| Trp (Boc) | tryptyl (t-butoxycarbonyl) |
| Tyr | tyrosyl |
| Tyr (t-Bu) | tyrosyl (O-t-butyl) |
| Val | valyl |

When not found in the table above, nomenclature and abbreviations may be further clarified by reference to the Calbiochem-Novabiochem Corp. 1999 Catalog and Peptide Synthesis Handbook or the Chem-Impex International, Inc. Tools for Peptide & Solid Phase Synthesis 1998-1999 Catalogue.

Determination of Biological Activity

In Vitro Assay for Angiogenic Activity

The human microvascular endothelial cell (HMVEC) migration assay was run according to the procedure of S. S. Tolsma, O. V. Volpert, D. J. Good, W. F. Frazier, P. J. Polverini and N. Bouck, J. Cell Biol. 1993, 122, 497-511.

The HMVEC migration assay was carried out using Human Microvascular Endothelial Cells-Dermal (single donor) and Human Microvascular Endothelial Cells, (neonatal). The BCE or HMVEC cells were starved overnight in DME containing 0.1% bovine serum albumin (BSA). Cells were then harvested with trypsin and resuspended in DME with 0.1% BSA at a concentration of 1.5×10⁶ cells per mL. Cells were added to the bottom of a 48 well modified Boyden chamber (Nucleopore Corporation, Cabin John, Md.). The chamber was assembled and inverted, and cells were allowed to attach for 2 hours at 37° C. to polycarbonate chemotaxis membranes (5 μm pore size) that had been soaked in 0.1% gelatin overnight and dried. The chamber was then reinverted, and test substances (total volume of 50 μL), including activators, 15 ng/mL bFGF/VEGF, were added to the wells of the upper chamber. The apparatus was incubated for 4 hours at 37° C. Membranes were recovered, fixed and stained (Diff Quick, Fisher Scientific) and the number of cells that had migrated to the upper chamber per 3 high power fields counted. Background migration to DME+0.1 BSA was subtracted and the data reported as the number of cells migrated per 10 high power fields (400×) or, when results from multiple experiments were combined, as the percent inhibition of migration compared to a positive control.

The compounds of the present invention inhibited human endothelial cell migration in the above assay by >68% at a concentration of 10 nM. Preferred compounds had percent inhibition values of >55% at a concentration of 0.1 nM, and most preferred compounds had percent inhibition values of >70% at a concentration of 0.1 nM. As shown by these results, the compounds of the present invention provide enhanced potency relative to the antiangiogenic peptides described in the art.

As shown by these results, the compounds of the invention inhibit migration of human endothelial cells, which is the first event in the angiogenesis process. As angiogenesis inhibitors, such compounds are useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compounds may also be useful in treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, these compounds may be useful in the prevention of metastases from the tumors described above either when used alone or in combination with radiotherapy and/or other chemotherapeutic agents.

Further uses include the treatment and prophylaxis of autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemagiomas, and capillary proliferation within atherosclerotic plaques; Osler-Webber Syndrome; myocardial angiogenesis; plaque neovascularization; telangiectasia; hemophiliac joints; angiofibroma; and wound granulation. Other uses include the treatment of diseases characterized by excessive or abnormal stimulation of endothelial cells, including but not limited to intestinal adhesions, Crohn's disease, atherosclerosis, scleroderma, and hypertrophic scars, i.e. keloids. Another use is as a birth control agent, by inhibiting ovulation and establishment of the placenta. The compounds of the invention are also useful in the treatment of diseases that have angiogenesis as a pathologic consequence such as cat scratch disease (Rochele minalia quintosa) and ulcers (Helicobacter pylori). The compounds of the invention are also useful to reduce bleeding by administration prior to sugery, especially for the treatment of resectable tumors.

The compounds of the invention may be used in combination with other compositions and procedures for the treatment of diseases. For example, a tumor may be treated conventionally with surgery, radiation or chemotherapy combined with a peptide of the present invention and then a peptide of the present invention may be subsequently administered to the patient to extend the dormancy of micrometastases and to stabilize and inhibit the growth of any residual primary tumor. Additionally, the compounds of the invention may be combined with pharmaceutically acceptable excipients, and optionally sustained-release matrices, such as biodegradable polymers, to form therapeutic compositions.

A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

When used in the above or other treatments, a therapeutically effective amount of one of the compounds of the present invention may be employed in pure form or, where such forms exist, in pharmaceutically acceptable salt form. By a “therapeutically effective amount” of the compound of the invention is meant a sufficient amount of the compound to treat an angiogenic disease, (for example, to limit tumor growth or to slow or block tumor metastasis) at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved.

Alternatively, a compound of the present invention may be administered as pharmaceutical compositions containing the compound of interest in combination with one or more pharmaceutically acceptable excipients. A pharmaceutically acceptable carrier or excipient refers to a non-toxic solid, semi-solid or liquid filler, diluent, encapsulating material or formulation auxiliary of any type. The compositions may be administered parenterally, intracisternally, intravaginally, intraperitoneally, topically (as by powders, ointments, drops or transdermal patch), rectally, or bucally. The term “parenteral” as used herein refers to modes of administration which include intravenous, intramuscular, intraperitoneal, intrasternal, subcutaneous and intraarticular injection and infusion.

Pharmaceutical compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Injectable depot forms are made by forming microencapsule matrices of the drug in biodegradable polymers such as polylactide-polyglycolide, poly(orthoesters), poly(anhydrides), and (poly)glycols, such as PEG. Depending upon the ratio of drug to polymer and the nature of the particular polymer employed, the rate of drug release can be controlled. Depot injectable formulations are also prepared by entrapping the drug in liposomes or microemulsions which are compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical administration, including those for inhalation, may be prepared as a dry powder which may be pressurized or non-pressurized. In non-pressurized powder compositions, the active ingredient in finely divided form may be used in admixture with a larger-sized pharmaceutically-acceptable inert carrier comprising particles having a size, for example, of up to 100 micrometers in diameter. Suitable inert carriers include sugars such as lactose. Desirably, at least 95% by weight of the particles of the active ingredient have an effective particle size in the range of 0.01 to 10 micrometers.

Alternatively, the composition may be pressurized and contain a compressed gas, such as nitrogen or a liquified gas propellant. The liquified propellant medium and indeed the total composition is preferably such that the active ingredient does not dissolve therein to any substantial extent. The pressurized composition may also contain a surface active agent, such as a liquid or solid non-ionic surface active agent or may be a solid anionic surface active agent. It is preferred to use the solid anionic surface active agent in the form of a sodium salt.

A further form of topical administration is to the eye. A compound of the invention is delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds of the invention may be injected directly into the vitreous and aqueous humour.

Compositions for rectal or vaginal administration are preferably suppositories which may be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

While the compounds of the invention can be administered as the sole active pharmaceutical agent, they may also be used in combination with one or more agents which are conventionally administered to patients for treating angiogenic diseases. For example, the compounds of the invention are effective over the short term to make tumors more sensitive to traditional cytotoxic therapies such as chemicals and radiation. The compounds of the invention also enhance the effectiveness of existing cytotoxic adjuvant anti-cancer therapies. The compounds of the invention may also be combined with other antiangiogenic agents to enhance their effectiveness, or combined with other antiangiogenic agents and administered together with other cytotoxic agents. In particular, when used in the treatment of solid tumors, compounds of the invention may be administered with IL-12, retinoids, interferons, angiostatin, endostatin, thalidomide, thrombospondin-1, thrombospondin-2, captopryl, angioinhibins, TNP-470, pentosan polysulfate, platelet factor 4, LM-609, SU-5416, CM-101, Tecogalan, plasminogen-K-5, vasostatin, vitaxin, vasculostatin, squalamine, marimastat or other MMP inhibitors, anti-neoplastic agents such as alpha inteferon, COMP (cyclophosphamide, vincristine, methotrexate and prednisone), etoposide, MBACOD (methortrexate, bleomycin, doxorubicin, cyclophosphamide, vincristine and dexamethasone), PRO-MACE/MOPP (prednisone, methotrexate (w/leucovin rescue), doxorubicin, cyclophosphamide, cisplatin, taxol, etoposide/mechlorethamine, vincristine, prednisone and procarbazine), vincristine, vinblastine, and the like as well as with radiation.

Total daily dose of the compositions of the invention to be administered to a human or other mammal host in single or divided doses may be in amounts, for example, from 0.0001 to 300 mg/kg body weight daily and more usually 1 to 300 mg/kg body weight.

It will be understood that agents which can be combined with the compound of the present invention for the inhibition, treatment or prophylaxis of angiogenic diseases are not limited to those listed above, but include in principle any agents useful for the treatment or prophylaxis of angiogenic diseases.

The peptides of the invention may be used for the development of affinity columns for isolation of receptors relevant to the antiangiogenic activity of the peptide of the invention, e.g. TSP-1 receptor, in, for example, cultured endothelial cells. As is known in the art, isolation and purification of the receptor may be followed by amino acid sequencing to identify and isolate polynucleotides which encode the receptor. Recombinant expression of this receptor would allow greater amounts of receptor to be produced, e.g. to produce a sufficient quantity for use in high throughput screening assays to identify other angiogenesis inhibitors.

Synthesis of the Peptides

The polypeptides of the present invention may be synthesized by any techniques that are known to those skilled in the art. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Acacemic Press (New York), 1965.

Reagents, resins, amino acids, and amino acid derivatives are commercially available and can be purchased from Chem-Impex International, Inc. (Wood Dale, Ill., U.S.A.) or Calbiochem-Novabiochem Corp. (San Diego, Calif., U.S.A.) unless otherwise noted herein.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

A particularly preferred method of preparing compounds of the present invention involves solid phase peptide synthesis.

In this particularly preferred method the α-amino function is protected by an acid or base sensitive group. Such protecting groups should have the properties of being stable to the conditions of peptide linkage formation, while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyl-oxycarbonyl, t-amyloxycarbonyl, isobornyloxycarbonyl, (α,α)-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is preferred.

Particularly preferred side chain protecting groups are as follows: for arginine and lysine: acetyl (Ac), and 2,2,5,7,8-pentamethylchroman-6-sulfonyl (Pmc); for asparagine: trityl (Trt); for glutamine: trityl (Trt); for serine: t-butyl (t-Bu); for threonine and allothreonine: t-butyl (t-Bu); for tryptophan: t-butoxycarbonyl (Boc); and for tyrosine: t-butyl (t-Bu).

In the solid phase peptide synthesis method, the C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. The preferred solid support for synthesis of C-terminal carboxy peptides is 4-hydroxymethyl-phenoxymethyl-copoly(styrene-1% divinylbenzene). The preferred solid support for C-terminal amide peptides is 4-(2′,4′-dimethoxyphenyl-Fmoc-aminomethyl)phenoxyacetamidoethyl resin available from Applied Biosystems.

The C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° and 50° C. in a solvent such as dichloromethane or N,N-dimethylformamide (DMF). When the solid support is 4-(2′,4′-dimethoxyphenyl-Fmoc-aminomethyl)-phenoxyacetamidoethyl resin, the Fmoc group is cleaved with a secondary amine, preferably piperidine, prior to coupling with the C-terminal amino acid as described above. The preferred method for coupling to the deprotected 4-(2′,4′-dimethoxyphenyl-Fmoc-aminomethyl)phenoxyacetamidoethyl resin is is O-benzotriazol-1-yl-N,N,N′,N′-tetramethyluroniumhexafluorophosphate (HBTU, 1 equiv.) and 1-hydroxybenzotriazole (HOBT, 1 equiv.) in N,N-dimethylformamide (DMF).

The coupling of successive protected amino acids can be carried out in an automatic polypeptide synthesizer as is well known in the art. In a preferred embodiment, the α-amino function in the amino acids of the growing peptide chain are protected with Fmoc. The removal of the Fmoc protecting group from the N-terminal side of the growing peptide is accomplished by treatment with a secondary amine, preferably piperidine. Each protected amino acid is then introduced in about 3-fold molar excess and the coupling is preferably carried out in N,N-dimethylformamide (DMF). The coupling agent is normally O-benzotriazol-1-yl-N,N,N′,N′-tetramethyluroniumhexafluorophosphate (HBTU, 1 equiv.) and 1-hydroxy-benzotriazole (HOBT, 1 equiv.).

At the end of the solid phase synthesis, the polypeptide is removed from the resin and deprotected, either in succession or in a single operation. Removal of the polypeptide and deprotection can be accomplished in a single operation by treating the resin-bound polypeptide with a cleavage reagent, for example thianisole, water, ethanedithiol and trifluoroacetic acid.

In cases wherein the C-terminus of the polypeptide is an alkylamide, the resin is cleaved by aminolysis with an alkylamine. Alternatively, the peptide may be removed by transesterification, e.g. with methanol, followed by aminolysis or by direct transamidation. The protected peptide may be purified at this point or taken to the next step directly. The removal of the side chain protecting groups is accomplished using the cleavage cocktail described above.

The fully deprotected peptide is purified by a sequence of chromatographic steps employing any or all of the following types: ion exchange on a weakly basic resin in the acetate form; hydrophobic adsorption chromatography on underivitized polystyrene-divinylbenzene (for example, AMBERLITE® XAD); silica gel adsorption chromatography; ion exchange chromatography on carboxymethylcellulose; partition chromatography, e.g. on SEPHADEX® G-25, LH-20 or countercurrent distribution; high performance liquid chromatography (HPLC), especially reverse-phase HPLC on octyl- or octadecylsilyl-silica bonded phase column packing.

The foregoing may be better understood in light of the following examples. Abbreviations which have been used in the following examples are: NMP for N-methylpyrrolidinone; HBTU for 2-(1H-benzotriazole-1-yl)-1,1,3,3-tetramethyluronium hexafluorophosphate; DMF for N,N-dimethylformamide; TFA for trifluoroacetic acid; and DMA for N,N-dimethylacetamide.

### EXAMPLE 1

### N-Ac-Sar-Gly-Gln-D-Ile-Thr-Nva-Ile-Arg-Pro-D-AlaNH₂

In the reaction vessel of an Applied Biosystems 433A peptide synthesizer was placed 0.1 mM of Fmoc-D-Ala-Sieber amide resin. Cartridges of 1 MM amino acids were sequentially loaded. The Fastmoc 0.1 with previous peak monitoring protocol was used with the following synthetic cycle:

1. Solvating resin with NMP for about 5 minutes;

2. Resin washed with NMP for about 5 minutes;

3. Fmoc group removed using 50% piperidine solution in NMP for 5 minutes, resin washed, and the sequence repeated 3 to 4 times;

4. Fmoc-amino acid activated with 1 mM of 0.5M of HBTU in DMF;

5. Activated Fmoc-amino acid added to the reaction vessel followed by addition of 1 mM of 2M diisopropylamine in NMP;

6. Fmoc-amino acid coupled for 20 minutes;

7. Resin washed and Fmoc-group removed using 50% piperidine in NMP.

The following protected amino acids were sequentially coupled to the resin using above protocol:

TABLE 2

| Amino acid | Coupling time |
|---|---|
| 1. Fmoc-Pro | 20 minutes |
| 2. Fmoc-Arg (Pmc) | 20 minutes |
| 3. Fmoc-Ile | 20 minutes |
| 4. Fmoc-Nva | 20 minutes |
| 5. Fmoc-Thr (t-Bu) | 20 minutes |
| 6. Fmoc-D-Ile | 20 minutes |
| 7. Fmoc-Gln (Trt) | 20 minutes |
| 8. Fmoc-Gly | 20 minutes |
| 9. Fmoc-Sar | 20 minutes |
| 10. acetic acid | 20 minutes |

Upon completion of the synthesis the resin-bound peptide was washed with methanol three times, dried in vacuo, then treated with a 95:5 TFA/water solution (3 mL) at room temperature overnight. The resin was filtered and washed 3 times with methanol. The filtrates and the washes were combined and concentrated in vacuo. The residue was treated with diethyl ether and the precipitate was filtered to provide the crude peptide as an amorphous powder. This was purified by preparative HPLC using a C-18 column with a solvent system increasing in gradient from 5% to 100% acetonitrile/water containing 0.01% TFA over a period of 50 minutes. The pure fractions were lyophilized to provide N-Ac-Sar-Gly-Gln-D-Ile-Thr-Nva-Ile-Arg-Pro-D-AlaNH₂ as the trifluoroacetate salt; Rₜ=2.15 minutes (10% to 40% acetonitrile in water containing 0.01% TFA over a period of 30 minutes); MS (ESI) m/e 1066 (M+H)⁺; Amino Acid Anal.: 0.89 Sar; 0.94 Gly; 0.94 Glu; 2.06 Ile; 0.50 Thr; 1.08 Nva; 1.11 Arg; 1.03 Pro; 1.02 Ala.

### EXAMPLE 2

### N-Ac-Sar-Gly-Phe-D-Ile-Thr-Nva-Ile-Arg-Pro-D-AlaNH₂

The desired compound was prepared by substituting Fmoc-Phe for Fmoc-Gln(Trt) in Example 1. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Phe-D-Ile-Thr-Nva-Ile-Arg-Pro-D-AlaNH₂ as the trifluoroacetate salt: Rₜ=3.565 minutes (10% to 40% acetonitrile in water containing 0.01% of TFA, over a period of 30 minutes); MS (ESI) m/e 1085 (M+H)⁺; Amino Acid Anal.: 0.88 Sar; 1.01 Gly; 0.91 Phe; 1.97 Ile; 0.31 Thr; 0.89 Nva; 1.06 Arg; 1.05 Pro; 1.03 Ala.

### EXAMPLE 3

### N-Ac-Sar-Gln-Val-D-Ile-Thr-Nva-Ile-Arg-ProNHCH₂CH₃

In the reaction vessel of an Applied Biosystems 433A peptide synthesizer was placed 0.1 mM of Fmoc-Pro-Sieber ethylamide resin. Cartridges of 1 mM amino acids were sequentially loaded. The Fastmoc 0.1 with previous peak monitoring protocol was used with the following synthetic cycle:

1. Solvating resin with NMP for about 5 minutes;

2. Resin washed with NMP for about 5 minutes;

3. Fmoc group removed using 50% piperidine solution in NMP for 5 minutes, resin washed, and the sequence repeated 3 to 4 times;

4. Fmoc-amino acid activated with 1 mM of 0.5M of HBTU in DMF;

5. Activated Fmoc-amino acid added to the reaction vessel followed by addition of 1 mM of 2M diisopropylamine in NMP;

6. Fmoc-amino acid coupled for 20 minutes;

7. Resin washed and Fmoc-group removed using 50% piperidine in NMP.

The following protected amino acids were sequentially coupled to the resin using above protocol:

TABLE 3

| Amino acid | Coupling time |
|---|---|
| 1. Fmoc-Arg (Pmc) | 20 minutes |
| 2. Fmoc-Ile | 20 minutes |
| 3. Fmoc-Nva | 20 minutes |
| 4. Fmoc-Thr (t-Bu) | 20 minutes |
| 5. Fmoc-D-Ile | 20 minutes |
| 6. Fmoc-Val | 20 minutes |
| 7. Fmoc-Gln (Trt) | 20 minutes |
| 8. Fmoc-Sar | 20 minutes |
| 9. acetic acid | 20 minutes |

Upon completion of the synthesis the resin-bound peptide was washed with methanol three times, dried in vacuo, then treated with a 95:5 TFA/water solution (3 mL) at room temperature overnight. The resin was filtered and washed 3 times with methanol. The filtrates and the washes were combined and concentrated in vacuo. The residue was treated with diethyl ether and the precipitate was filtered to provide the crude peptide as an amorphous powder. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gln-Val-D-Ile-Thr-Nva-Ile-Arg-ProNHCH₂CH₃ as the trifluoroacetate salt: Rₜ=3.06 minutes (10% to 40% acetonitrile in water containing 0.01% of TFA, over a period of 30 minutes); MS (ESI) m/e 1065 (M+H)⁺; Amino Acid Anal.: 0.92 Sar; 0.90 Gln; 1.01 Val; 2.07 Ile; 0.57 Thr; 1.03 Nva; 1.36 Arg; 1.10 Pro.

### EXAMPLE 4

### N-Ac-Sar-Gly-Val-D-Ile-alloThr-Nva-Ile-Arg-ProNHCH₂CH₃

The desired compound was prepared by substituting Fmoc-Gly and Fmoc-alloThr(t-Bu) for Fmoc-Gln(Trt) and Fmoc-Thr(t-Bu), respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-Ile-alloThr-Nva-Ile-Arg-ProNHCH₂CH₃ as the trifluoroacetate salt: Rₜ=3.52 minutes (10% to 40% acetonitrile in water containing 0.01% of TFA, over a 30 minute period); MS (ESI) m/e 994 (M+H)⁺; Amino Acid Anal.: 1.01 Sar; 1.00 Gly; 0.97 Val; 2.10 Ile; 0.58 Thr; 0.98 Nva; 1.0 Arg; 1.07 Pro.

### EXAMPLE 5

### N-Ac-Sar-Gly-Val-D-Ile-Thr-Nva-D-Ile-Arg-ProNHCH₂CH₃

The desired compound was prepared by substituting Fmoc-Gly and Fmoc-D-Ile for Fmoc-Gln(Trt) and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-Ile-Thr-Nva-D-Ile-Arg-ProNHCH₂CH₃ as the trifluoroacetate salt: Rₜ=3.73 minutes (10% to 40% acetonitrile in water containing 0.01% of TFA, over a 30 minute period); MS (ESI) m/e 994 (M+H)⁺; Amino Acid Anal.: 1.03 Sar; 0.99 Gly; 0.97 Val; 2.11 Ile; 0.45 Thr; 1.04 Nva; 0.97 Arg; 1.04 Pro.

### EXAMPLE 6

### N-Ac-Sar-Gly-Asn-D-Leu-Ser-Nva-Ile-Arg-ProNHCH₂CH₃

The desired compound was prepared by substituting Fmoc-Gly, Fmoc-Asn(Trt), Fmoc-D-Leu, and Fmoc-Ser(t-Bu) for Fmoc-Gln(Trt), Fmoc-Val, Fmoc-D-Ile, and Fmoc-Thr(t-Bu), respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Asn-D-Leu-Ser-Nva-Ile-Arg-ProNHCH₂CH₃ as the trifluoroacetate salt: Rₜ=2.798 minutes (10% to 40% acetonitrile in water containing 0.01% of TFA, over a 30 minute period); MS (ESI) m/e 995 (M+H)⁺; Amino Acid Anal.: 0.94 Sar; 0.98 Gly; 0.99 Asp; 1.05 Leu; 0.26 Ser; 0.94 Nva; 0.95 Ile; 1.0 Arg; 1.09 Pro.

### EXAMPLE 7

### N-(6-Me-Nicotinyl)-Sar-Gly-Val-D-Ile-Thr-Nva-Ile-Arg-ProNHCH₂CH₃

The desired compound was prepared by substituting 6-methylnicotinic acid and Fmoc-Gly for acetic acid and Fmoc-Gln(Trt), respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-(6-Me-Nicotinyl)-Sar-Gly-Val-D-Ile-Thr-Nva-D-Ile-Arg-ProNHCH₂CH₃ as the trifluoroacetate salt: Rₜ=3.82 minutes (10% to 40% acetonitrile in water containing 0.01% of TFA, over a 30 minute period); MS (ESI) m/e 1071 (M+H)⁺; Amino Acid Anal.: 0.98 Sar; 1.02 Gly; 1.01 Val; 2.05 Ile; 0.51 Thr; 1.01 Nva; 1.00 Arg; 1.08 Pro.

### EXAMPLE 8

### N-Ac-Sar-Gly-Val-Ile-Thr-Nva-D-Ile-Arg-ProNHCH₂CH₃

The desired compound was prepared by substituting Fmoc-Gly, Fmoc-Ile, and Fmoc-D-Ile for Fmoc-Gln(Trt), Fmoc-D-Ile, and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-Ile-Thr-Nva-D-Ile-Arg-ProNHCH₂CH₃ as the trifluoroacetate salt: Rₜ=3.62 minutes (10% to 40% acetonitrile in water containing 0.01% of TFA, over a 30 minute period); MS (ESI) m/e 994 (M+H)⁺; Amino Acid Anal.: 1.03 Sar; 1.03 Gly; 0.96 Val; 2.12 Ile; 0.43 Thr; 1.02 Nva; 1.02 Arg; 1.02 Pro.

### EXAMPLE 9

### N-Ac-Sar-Gly-Val-D-alloIle-Ser-Thr-Ile-Arg-ProNHCH₂CH₃

The desired compound was prepared by substituting Fmoc-Gly, Fmoc-D-alloIle, Fmoc-Ser(t-Bu), and Fmoc-Thr(t-Bu) for Fmoc-Gln(Trt), Fmoc-D-Ile, Fmoc-Thr(t-Bu), and Fmoc-Nva, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-alloIle-Ser-Thr-Ile-Arg-ProNHCH₂CH₃ as the trifluoroacetate salt: Rₜ=3.089 minutes (10% to 40% acetonitrile in water containing 0.01% of TFA, over a 30 minute period); MS (ESI) m/e 982 (M+H)⁺; Amino Acid Anal.: 0.95 Sar; 0.96 Gly; 0.99 Val; 1.06 alloIle; 0.97 Ile; 0.57 Thr; 0.31 Ser; 1.02 Arg; 1.02 Pro.

### EXAMPLE 10

### N-Ac-Sar-Gly-Gln-D-Ile-Thr-Nva-D-Ile-Arg-ProNHCH₂CH₃

The desired compound was prepared by substituting Fmoc-Gly, Fmoc-Gln(Trt), and Fmoc-D-Ile for Fmoc-Gln(Trt), Fmoc-Val, and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Gln-D-Ile-Thr-Nva-D-Ile-Arg-ProNHCH₂CH₃ as the trifluoroacetate salt: Rₜ=2.89 minutes (10% to 40% acetonitrile in water containing 0.01% of TFA, over a 30 minute period); MS (ESI) m/e 1023 (M+H)⁺; Amino Acid Anal.: 1.00 Sar; 0.97 Gly; 0.93 Glu; 2.15 Ile; 0.57 Thr; 1.02 Nva; 1.11 Arg; 1.10 Pro.

### EXAMPLE 11

### N-Ac-Sar-Gly-Asn-D-Ile-Thr-Nva-Lys(Ac)-Arg-ProNHCH₂CH₃

The desired compound was prepared by substituting Fmoc-Gly, Fmoc-Asn(Trt), and Fmoc-Lys(Ac) for Fmoc-Gln(Trt), Fmoc-Val, and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent mixture varying in a gradient from 5% to 100% acetonitrile/water containing 0.01% TFA over a period of 50 minutes. The pure fractions were lyophilized to provide N-Ac-Sar-Gly-Asn-D-Ile-Thr-Nva-Lys(Ac)-Arg-ProNHCH₂CH₃ as the trifluoroacetate salt: Rₜ=4.58 minutes (10% to 40% acetonitrile in water containing 0.01% of TFA, over a 30 minute period); MS (ESI) m/e 1066 (M+H)⁺; Amino Acid Anal.: 0.98 Sar; 0.96 Gly; 0.92 Asp; 1.01. Ile; 0.52 Thr; 1.04 Nva; 1.03 Lys; 0.95 Arg; 1.06 Pro.

### EXAMPLE 12

### N-Ac-Sar-Gly-Gln-D-alloIle-Tyr-Nva-D-Ile-Arg-ProNHCH₂CH₃

The desired compound was prepared by substituting Fmoc-Gly, Fmoc-Gln(Trt), Fmoc-D-alloIle, Fmoc-Tyr(t-Bu), and Fmoc-D-Ile for Fmoc-Gln(Trt), Fmoc-Val, Fmoc-D-Ile, Fmoc-Thr(t-Bu), and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Gln-D-alloIle-Tyr-Nva-D-Ile-Arg-ProNHCH₂CH₃ as the trifluoroacetate salt: Rₜ=3.357 minutes (10% to 40% acetonitrile in water containing 0.01% of TFA, over a 30 minute period); MS (ESI) m/e 1085 (M+H)⁺; Amino Acid Anal.: 0.93 Sar; 0.97 Gly; 0.99 Glu; 2.07 Ile; 0.93 Tyr; 1.01 Nva; 0.97 Arg; 1.00 Pro.

### EXAMPLE 13

### N-Ac-Sar-Gly-Gln-D-alloIle-Thr-Nva-Ile-Arg-Pro-D-AlaNH₂

The desired compound was prepared by substituting Fmoc-D-alloIle for Fmoc-D-Ile in Example 1. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Gln-D-alloIle-Thr-Nva-Ile-Arg-Pro-D-AlaNH₂ as the trifluoroacetate salt: Rₜ=2.38 minutes (10% to 40% acetonitrile in water containing 0.01% of TFA, over a 30 minute period); MS (ESI) m/e 1066 (M+H)⁺; Amino Acid Anal.: 0.99 Sar; 1.00. Gly; 0.83 Glu; 2.03 Ile; 0.47 Thr; 1.02 Nva; 1.05 Arg; 1.03 Pro; 1.03 Ala.

### EXAMPLE 14

### N-Ac-Sar-Gly-Asn-D-Leu-Thr-Ser-Ile-Arg-ProNHCH₂CH₃

The desired compound was prepared by substituting Fmoc-Gly, Fmoc-Asn(Trt), Fmoc-D-Leu, and Fmoc-Ser(t-Bu) for Fmoc-Gln(Trt), Fmoc-Val, Fmoc-D-Ile, and Fmoc-Nva, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Asn-D-Leu-Thr-Ser-Ile-Arg-ProNHCH₂CH₃ as the trifluoroacetate salt: Rₜ=2.375 minutes (10% to 40% acetonitrile in water containing 0.01% of TFA, over a 30 minute period); MS (ESI) m/e 997 (M+H)⁺; Amino Acid Anal.: 0.93 Sar; 0.98 Gly; 0.96 Asp; 1.03 Leu; 0.54 Thr; 0.21 Ser; 0.97 Ile; 1.01 Arg; 1.06 Pro.

### EXAMPLE 15

### N-Ac-Sar-Gly-Val-D-Ile-alloThr-Ser-Ile-Arg-ProNHCH₂CH₃

The desired compound was prepared by substituting Fmoc-Gly, Fmoc-alloThr(t-Bu), and Fmoc-Ser(t-Bu) for Fmoc-Gln(Trt), Fmoc-Thr(t-Bu), and Fmoc-Nva, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-Ile-alloThr-Ser-Ile-Arg-ProNHCH₂CH₃ as the trifluoroacetate salt: Rₜ=3.104 minutes (10% to 40% acetonitrile in water containing 0.01% of TFA, over a 30 minute period); MS (ESI) m/e 982 (M+H)⁺; Amino Acid Anal.: 0.97 Sar; 1.00 Gly; 0.71 Val; 1.64 Ile; 0.54 Thr; 0.20 Ser; 1.08 Arg; 1.02 Pro.

### EXAMPLE 16

### N-Ac-Sar-Gly-Gln-D-Ile-alloThr-Nva-Ile-Arg-ProNHCH₂CH₃

The desired compound was prepared by substituting Fmoc-Gly, Fmoc-Gln(Trt), and Fmoc-alloThr(t-Bu) for Fmoc-Gln(Trt), Fmoc-Val, and Fmoc-Thr(t-Bu), respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Gln-D-Ile-alloThr-Nva-Ile-Arg-ProNHCH₂CH₃ as the trifluoroacetate salt: Rₜ=2.835 minutes (10% to 40% acetonitrile in water containing 0.01% of TFA, over a 30 minute period); MS (ESI) m/e 1023 (M+H)⁺; Amino Acid Anal.: 0.98 Sar; 0.99 Gly; 1.05 Glu; 1.93 Ile; 0.64 Thr; 0.92 Nva; 1.12 Arg; 1.12 Pro.

### EXAMPLE 17

### N-Ac-Sar-Gly-Val-D-Ile-alloThr-Nva-Pro-Arg-ProNHCH₂CH₃

The desired compound was prepared by substituting Fmoc-Gly, Fmoc-alloThr(t-Bu), and Fmoc-Pro for Fmoc-Gln(Trt), Fmoc-Thr(t-Bu), and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-Ile-alloThr-Nva-Pro-Arg-ProNHCH₂CH₃ as the trifluoroacetate salt: Rₜ=3.149 minutes (10% to 40% acetonitrile in water containing 0.01% of TFA, over a 30 minute period); MS (ESI) m/e 978 (M+H)⁺; Amino Acid Anal.: 0.90 Sar; 0.99 Gly; 1.03 Glu; 0.89 Ile; 0.59 Thr; 0.85 Nva; 1.16 Arg; 2.14 Pro.

### EXAMPLE 18

### N-Ac-Sar-Gly-Val-D-alloIle-Thr-Trp-Ile-Arg-ProNHCH₂CH₃

The desired compound was prepared by substituting Fmoc-Gly, Fmoc-D-alloIle, and Fmoc-Trp(Boc) for Fmoc-Gln(Trt), Fmoc-D-Ile, and Fmoc-Nva, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-alloIle-Thr-Trp-Ile-Arg-ProNHCH₂CH₃ as the trifluoroacetate salt: Rₜ=4.203 minutes (10% to 40% acetonitrile in water containing 0.01% of TFA, over a 30 minute period); MS (ESI) m/e 1081 (M+H)⁺; Amino Acid Anal.: 0.91 Sar; 0.96 Gly; 0.99 Val; 1.04 alloIle; 0.61 Thr; 0.21 Trp; 1.03 Arg; 1.03 Pro.

### EXAMPLE 19

### N-Ac-Sar-Gly-Val-D-alloIle-Ser-Ser-Ile-Arg-ProNHCH(CH₃)₂

Resin Preparation

4-(4-Formyl-3-methoxyphenoxy)butyryl AM resin (0.5 g, 0.54 mmol/g substitution) was placed in a solid phase synthesis reaction vessel containing 9:1 DMA/acetic acid (4 mL). The mixture was shaken for 5 minutes, the resin was drained, and the process was repeated three times. The swollen resin was treated with 10-15 grains of activated 4 Å molecular sieves, 9:1 DMA/acetic acid (4 mL), and 10 equivalents of isopropylamine. The resulting slurry was shaken at room temperature for 1 hour, treated with 10 equivalents of sodium triacetoxyborohydride, and shaken for 2 hours. The resin was drained, washed three times with DMA, three times with methanol, three times with dichloromethane, three times with diethyl ether, and dried under vacuum for 16 hours. The dry resin was treated with DMA (4 mL), shaken for 5 minutes, and the process was repeated two times.

Coupling of Fmoc-Pro

The resin was treated sequentially with DMA (4 mL), diisopropylethylamine (1 equivalent), Fmoc-Pro (3 equivalents ) in DMA, HATU (3 equivalents), and diisopropylethylamine (3 equivalents), and shaken for 16 hours. The resin was drained, washed three times with DMA, three times with methanol, three times with dichloromethane, three times with diethyl ether, and dried under vacuum for 16 hours. The resin was treated with DMA (4 mL), shaken for 5 minutes, and the process was repeated three times. A solution of 8:1:1 DMA/pyridine/acetic anhydride (5 mL) was added and the resulting mixture was shaken for 1 hour. The resin was drained and washed three times with DMA, three times with methanol, three times with dichloromethane, and three times with diethyl ether. The resin was dried under vacuum at room temperature for 16 hours.

Synthesis of the Peptide

The desired compound was prepared by substituting the above resin, Fmoc-Gly, Fmoc-D-alloIle, Fmoc-Ser(t-Bu), and Fmoc-Ser(t-Bu) for Fmoc-Pro-Sieber ethylamide resin, FmocGln(Trt), Fmoc-D-Ile, Fmoc-Thr(t-Bu), and Fmoc-Nva, respectively, in Example 3. Upon completion of the synthesis the peptide and the protecting groups were cleaved with 95:5 TFA/anisole (3 mL) over 3 hours. The resin was filtered, washed three times with methanol, and concentrated. The residue was treated with diethyl ether and the resulting solid was filtered to provide the crude peptide. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-alloIle-Ser-Ser-Ile-Arg-ProNHCH₂(CH₃)₂ as the trifluoroacetate salt: Rₜ=2.88 minutes (10% to 40% acetonitrile in water containing 0.01% of TFA, over a 30 minute period); MS (ESI) m/e 982 (M+H)⁺; Amino Acid Anal.: 1.03 Sar; 0.97 Gly; 0.99 Val; 2.04 Ile; 0.78 Ser; 1.00 Arg; 1.06 Pro.

### EXAMPLE 20

### N-Ac-Sar-Gly-Val-D-Ile-Thr-Gln-D-Ile-Arg-ProNHCH₂CH₃

The desired compound was prepared by substituting Fmoc-Gly, Fmoc-Gln(Trt), and Fmoc-D-Ile for Fmoc-Gln(Trt), Fmoc-Nva, and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-Ile-Thr-Gln-D-Ile-Arg-ProNHCH₂CH₃ as the trifluoroacetate salt: Rₜ=3.01 minutes (10% to 40% acetonitrile in water containing 0.01% of TFA, over a 30 minute period); MS (ESI) m/e 1023 (M+H)⁺; Amino Acid Anal.: 1.00 Sar; 1.02 Gly; 1.03 Val; 2.11 Ile; 0.49 Thr; 0.92 Glu; 0.93 Arg; 1.04 Pro.

### EXAMPLE 21

### N-Ac-Sar-Gly-Val-D-alloIle-Thr-Trp-D-Ile-Arg-ProNHCH₂CH₃

The desired compound was prepared by substituting Fmoc-Gly, Fmoc-D-alloIle, Fmoc-Trp(Boc), and Fmoc-D-Ile for Fmoc-Gln(Trt), Fmoc-D-Ile, Fmoc-Nva, and Fmoc-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent mixture increasing in gradient from 5% to 100% acetonitrile/water containing 0.01% TFA over a period of 50 minutes. The pure fractions were lyophilized to provide N-Ac-Sar-Gly-Val-D-alloIle-Thr-Trp-D-Ile-Arg-ProNHCH₂CH₃ as the trifluoroacetate salt: Rₜ=4.44 minutes (10% to 40% acetonitrile in water containing 0.01% of TFA, over a 30 minute period); MS (ESI) m/e 1081 (M+H)⁺; Amino Acid Anal.: 1.05 Sar; 0.97 Gly; 0.96 Val; 2.05 Ile; 0.51 Thr; 0.28 Trp; 1.07 Arg; 1.09 Pro.

### EXAMPLE 22

### N-Ac-Sar-Gly-Val-D-alloIle-Thr-Nva-Ile-Arg-D-ProNHCH₂CH₃

The desired compound was prepared by substituting Fmoc-D-Pro-Sieber ethylamide resin, Fmoc-Gly, and Fmoc-D-alloIle for Fmoc-Pro-Sieber ethylamide resin, Fmoc-Gln(Trt), and Fmoc-D-Ile, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-alloIle-Thr-Nva-Ile-Arg-D-ProNHCH₂CH₃ as the trifluoroacetate salt: Rₜ=3.52 minutes (10% to 40% acetonitrile in water containing 0.01% of TFA, over a 30 minute period); MS (ESI) m/e 994 (M+H)⁺; Amino Acid Anal.: 1.00 Sar; 1.02 Gly; 1.01 Val; 2.10 Ile; 0.55 Thr; 0.99 Nva; 0.92 Arg; 1.01 Pro.

### EXAMPLE 23

### N-Ac-Sar-Gly-Val-D-Ile-Met-Nva-Ile-Arg-Pro-D-AlaNH₂

The desired compound was prepared by substituting Fmoc-Val and Fmoc-Met for Fmoc-Gln(Trt) and Fmoc-Thr(t-Bu), respectively, in Example 1. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-Ile-Met-Nva-Ile-Arg-Pro-D-AlaNH₂ as the trifluoroacetate salt: Rₜ=4.05 minutes (10% to 40% acetonitrile in water containing 0.01% of TFA, over a 30 minute period); MS (ESI) m/e 1067 (M+H)⁺; Amino Acid Anal.: 0.92 Sar; 0.96 Gly; 1.03 Val; 2.05 Ile; 0.91 Met; 1.05 Nva; 1.03 Arg; 1.02 Pro.

### EXAMPLE 24

### N-Ac-Sar-Gly-Val-D-Ile-alloThr-Pro-Ile-Arg-Pro-NHCH₂CH₃

The desired compound was prepared by substituting Fmoc-Gly, Fmoc-alloThr(t-Bu), and Fmoc-Pro for Fmoc-Gln(Trt), Fmoc-Thr(t-Bu), and Fmoc-Nva, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-Ile-alloThr-Nva-Pro-le-Arg-Pro-NHCH₂CH₃ as the trifluoroacetate salt: Rₜ=3.551 minutes (10% to 40% acetonitrile in water containing 0.01% of TFA, over a period of 30 minutes); MS (ESI) m/e 992 (M+H)⁺; Amino Acid Anal.: 0.95 Sar; 0.97 Gly; 0.99 Val; 1.96 Ile; 0.97 Arg; 2.08 Pro.

### EXAMPLE 25

### N-Ac-Sar-Gly-Val-D-alloIle-alloThr-Gln-Ile-Arg-Pro-NHCH₂CH₃

The desired compound was prepared by substituting Fmoc-Gly, Fmoc-D-alloIle, Fmoc-alloThr(t-Bu), and Fmoc-Gln(Trt) for Fmoc-Gln(Trt), Fmoc-D-Ile, Fmoc-Thr(t-Bu), and Fmoc-Nva, respectively, in Example 3. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent mixture varying in a gradient from 5% to 100% acetonitrile/water containing 0.01% TFA over a period of 50 minutes. The pure fractions were lyophilized to provide N-Ac-Sar-Gly-Val-D-alloIle-alloThr-Gln-Ile-Arg-Pro-NHCH₂CH₃ as the trifluoroacetate salt: Rₜ=3.08 minutes (10% to 40% acetonitrile in water containing 0.01% of TFA, over a period of 30 minutes); MS (ESI) m/e 1023 (M+H)⁺; Amino Acid Anal.: 0.95 Sar; 0.91 Gly; 1.00 Val; 2.02 Ile; 0.98 Glu; 1.01 Arg; 1.05 Pro.

### EXAMPLE 26

### N-Ac-Sar-Gly-Val-D-alloIle-Ser-Ser-Ile-Arg-Pro-D-AlaNH₂

The desired compound was prepared by substituting Fmoc-Val, Fmoc-D-alloIle, Fmoc-Ser(t-Bu), and Fmoc-Ser(t-Bu) for Fmoc-Gln(Trt), Fmoc-D-Ile, Fmoc-Thr(t-Bu), and Fmoc-Nva, respectively, in Example 1. Upon completion of the synthesis, cleavage of the peptide from the resin, removal of the protecting groups, precipitation with diethyl ether, and filtration, the crude peptide was obtained. This was purified by HPLC using a C-18 column and eluting with a solvent system increasing in gradient from 5% to 100% acetonitrile/water containing 0.01% TFA over a period of 50 minutes. The pure fractions were lyophilized to provide N-Ac-Sar-Gly-Val-D-alloIle-Ser-Ser-Ile-Arg-Pro-D-AlaNH₂ as the trifluoroacetate salt: Rₜ=2.80 minutes (10% to 40% acetonitrile in water containing 0.01% of TFA, over 30 minutes); MS (ESI) m/e 1011 (M+H)⁺; Amino Acid Anal.: 1.04 Sar; 0.99 Gly; 0.92 Val; 1.01 alloIle; 0.44 Ser; 0.95 Arg; 1.05 Pro.

What is claimed is:

1. A compound of formula (I)
N-Ac-Sar-Gly-AA³-AA⁴-AA⁵-AA⁶-AA⁷-Arg-Pro-AA¹⁰ (I),
or a pharmacutically acceptable salt, ester, prodrug, or solvate thereof, wherein
AA³ is selected from the group consisting of
(1) glutaminyl,
(2) phenylalanyl,
(3) valyl, and
(4) asparaginyl;
AA⁴ is selected from the group consisting of
(1) D-isoleucyl,
(2) isoleucyl,
(3) D-leucyl, and
(4) D-alloisoleucyl;
AA⁵ is selected from the group consisting of
(1) seryl,
(2) methionyl,
(3) allothreonyl,
(4) threonyl, and
(5) tyrosyl;
AA⁶ is selected from the group consisting of
(1) norvalyl,
(2) seryl,
(3) tryptophyl,
(4) glutaminyl, and
(5) prolyl;
AA⁷is selected from the group consisting of
(1) isoleucyl,
(2) D-isoleucyl,
(3) lysyl(acetyl), and
(4) prolyl; and
AA¹⁰ is selected from the group consisting of
(1) D-alanylamide,
(2) ethylamide, and
(3) isopropylamide;
with the proviso that one of AA⁴ and AA⁷ is a D-amino acid.

2. A compound according to claim 1 wherein AA⁴ is D-Ile.

3. A compound according to claim 2 selected from the group consisting of
N-Ac-Sar-Gly-Gln-D-Ile-Thr-Nva-Ile-Arg-Pro-D-AlaNH₂,
N-Ac-Sar-Gly-Phe-D-Ile-Thr-Nva-Ile-Arg-Pro-D-AlaNH₂,
N-Ac-Sar-Gly-Val-D-Ile-alloThr-Nva-Ile-Arg-ProNHCH₂CH₃,
N-Ac-Sar-Gly-Val-D-Ile-Thr-Nva-D-Ile-Arg-ProNHCH₂CH₃,
N-Ac-Sar-Gly-Gln-D-Ile-Thr-Nva-D-Ile-Arg-ProNHCH₂CH₃,
N-Ac-Sar-Gly-Asn-D-Ile-Thr-Nva-Lys(Ac)-Arg-ProNHCH₂CH₃,
N-Ac-Sar-Gly-Val-D-Ile-alloThr-Ser-Ile-Arg-ProNHCH₂CH₃,
N-Ac-Sar-Gly-Gln-D-Ile-alloThr-Nva-Ile-Arg-ProNHCH₂CH₃,
N-Ac-Sar-Gly-Val-D-Ee-alloThr-Nva-Pro-Arg-ProNHCH₂CH₃,
N-Ac-Sar-Gly-Val-D-Ile-Thr-Gln-D-Ile-Arg-ProNHCH₂CH₃,
N-Ac-Sar-Gly-Val-D-Ile-Met-Nva-Ile-Arg-Pro-D-AlaNH₂, and
N-Ac-Sar-Gly-Val-D-Ile-alloThr-Pro-Ile-Arg-ProNHCH₂CH₃.

4. A compound according to claim 1 wherein AA⁴ is D-Leu.

5. A compound according to claim 4 selected from the group consisting of
N-Ac-Sar-Gly-Asn-D-Leu-Ser-Nva-Ile-Arg-ProNHCH₂CH₃, and
N-Ac-Sar-Gly-Asn-D-Leu-Thr-Ser-Ile-Arg-ProNHCH₂CH₃.

6. A compound according to claim 1 wherein AA⁴ is D-alloIle.

7. A compound according to claim 6 selected from the group consisting of
N-Ac-Sar-Gly-Val-D-alloIle-Ser-Thr-Ile-Arg-ProNHCH₂CH₃,
N-Ac-Sar-Gly-Gln-D-alloIle-Tyr-Nva-D-Ile-Arg-ProNHCH₂CH₃,
N-Ac-Sar-Gly-Gln-D-alloIle-Thr-Nva-Ile-Arg-Pro-D-AlaNH₂,
N-Ac-Sar-Gly-Val-D-alloIle-Thr-Trp-Ile-Arg-ProNHCH₂CH₃,
N-Ac-Sar-Gly-Val-D-alloIle-Ser-Ser-Ile-Arg-ProNHCH(CH₃)₂,
N-Ac-Sar-Gly-Val-D-alloIle-Thr-Trp-D-Ile-Arg-ProNHCH₂CH₃,
N-Ac-Sar-Gly-Val-D-alloIle-alloThr-Gln-Ile-Arg-ProNHCH₂CH₃, and
N-Ac-Sar-Gly-Val-D-alloIle-Ser-Ser-Ile-Arg-Pro-D-AlaNH₂.

8. A pharmaceutical composition comprising a compound of claim 1, or a pharmacutically acceptable salt, ester, prodrug, or solvate thereof, and a pharmaceutically acceptable carrier.

9. A method of treating a patient in need of anti-angiogenesis therapy comprising administering to the patient in need a therapeutically effective amount of a compound in claim 1, or a pharmacutically acceptable salt, ester, prodrug, or solvate thereof.

10. A composition for the treatment of a disease selected from cancer, arthritis, psoriasis, angiogenesis of the eye associated with infection or surgical intervention, macular degeneration and diabetic retinopathy comprising a peptide as defined in claim 1, or a pharmacutically acceptable salt, ester, prodrug, or solvate thereof, in combination with a pharmaceutically acceptable carrier.

11. A method of isolating a receptor from an endothelial cell comprising binding a peptide as defined in claim 1, or a pharmacutically acceptable salt, ester, prodrug, or solvate thereof, to the receptor to form a peptide receptor complex; isolating the peptide receptor complex; and purifying the receptor.

12. A compound selected from the group consisting of
N-Ac-Sar-Gly-Gln-D-Ile-Thr-Nva-Ile-Arg-Pro-D-AlaNH₂,
N-Ac-Sar-Gly-Phe-D-Ile-Thr-Nva-Ile-Arg-Pro-D-AlaNH₂,
N-Ac-Sar-Gln-Val-D-Ile-Thr-Nva-Ile-Arg-ProNHCH₂CH₃,
N-Ac-Sar-Gly-Val-D-Ile-alloThr-Nva-Ile-Arg-ProNHCH₂CH₃,
N-Ac-Sar-Gly-Val-D-Ile-Thr-Nva-D-Ile-Arg-ProNHCH₂CH₃,
N-Ac-Sar-Gly-Asn-D-Leu-Ser-Nva-Ile-Arg-ProNHCH₂CH₃,
N-(6-Me-Nicotinyl)-Sar-Gly-Val-D-Ile-Thr-Nva-Ile-Arg-ProNHCH₂CH₃,
N-Ac-Sar-Gly-Val-Ile-Thr-Nva-D-Ile-Arg-ProNHCH₂CH₃,
N-Ac-Sar-Gly-Val-D-alloIle-Ser-Thr-Ile-Arg-ProNHCH₂CH₃,
N-Ac-Sar-Gly-Gln-D-Ile-Thr-Nva-D-Ile-Arg-ProNHCH₂CH₃,
N-Ac-Sar-Gly-Asn-D-Ile-Thr-Nva-Lys(Ac)-Arg-ProNHCH₂CH₃,
N-Ac-Sar-Gly-Gln-D-alloIle-Tyr-Nva-D-Ile-Arg-ProNHCH₂CH₃,
N-Ac-Sar-Gly-Gln-D-alloIle-Thr-Nva-Ile-Arg-Pro-D-AlaNH₂,
N-Ac-Sar-Gly-Asn-D-Leu-Thr-Ser-Ile-Arg-ProNHCH₂CH₃,
N-Ac-Sar-Gly-Val-D-Ile-alloThr-Ser-Ile-Arg-ProNHCH₂CH₃,
N-Ac-Sar-Gly-Gln-D-Ile-alloThr-Nva-Ile-Arg-ProNHCH₂CH₃,
N-Ac-Sar-Gly-Val-D-Ile-alloThr-Nva-Pro-Arg-ProNHCH₂CH₃,
N-Ac-Sar-Gly-Val-D-alloIle-Thr-Trp-Ile-Arg-ProNHCH₂CH₃,
N-Ac-Sar-Gly-Val-D-alloIle-Ser-Ser-Ile-Arg-ProNHCH(CH₃)₂,
N-Ac-Sar-Gly-Val-D-Ile-Thr-Gln-D-Ile-Arg-ProNHCH₂CH₃,
N-Ac-Sar-Gly-Val-D-alloIle-Thr-Trp-D-Ile-Arg-ProNHCH₂CH₃,
N-Ac-Sar-Gly-Val-D-alloIle-Thr-Nva-Ile-Arg-D-ProNHCH₂CH₃,
N-Ac-Sar-Gly-Val-D-Ile-Met-Nva-Ile-Arg-Pro-D-AlaNH₂,
N-Ac-Sar-Gly-Val-D-Ile-alloThr-Pro-Ile-Arg-ProNHCH₂CH₃,
N-Ac-Sar-Gly-Val-D-alloIle-alloThr-Gln-Ile-Arg-ProNHCH₂CH₃, and
N-Ac-Sar-Gly-Val-D-alloIle-Ser-Ser-Ile-Arg-Pro-D-AlaNH₂.
